(19) United States
(12) Patent Application Publication
(10) Pub. No.: US 2006/0035907 A1
(43) Pub. Date: Feb. 16, 2006

(54) Methods of treating abnormal cell growth using c-MET and m-TOR inhibitors

(76) Inventors: James G. Christensen, San Diego, CA (US); Ravi Salgia, Park Ridge, IL (US)

Correspondence Address: AGOURON PHARMACEUTICALS, INC., 10777 SCIENCE CENTER DRIVE, SAN DIEGO, CA, 92121, US

(21) Appl. No.: 11/063,033

(22) Filed: Feb. 22, 2005

Related U.S. Application Data
(60) Provisional application No. 60/546,850, filed on Feb. 23, 2004.

(51) Int. Cl. A61K 39/395; A61K 31/496; A61K 31/495; A61K 31/4745

(52) U.S. Cl. 514/254.09; 514/291; 424/146.1

(57) ABSTRACT

The invention provides a method of treating abnormal cell growth in a mammal, such as a human, by administering to the mammal a therapeutically effective amount of a c-MET inhibitor and a mammalian target of rapamycin (mTOR) inhibitor.

This application claims the benefit of U.S. Provisional Application Ser. No. 60/546,850, filed Feb. 23, 2004, the disclosure of which is incorporated herein by reference in its entirety.

### FIELD OF THE INVENTION

This invention relates to methods of treatment of abnormal cell growth, such as cancer, in mammals. In particular, the invention provides methods of treatment of abnormal cell growth using a c-MET inhibitor and an mTOR inhibitor.

### BACKGROUND

c-MET receptor tyrosine kinase (RTK) has been shown in many human cancers to be involved in oncogenesis, tumor progression with enhanced cell motility and invasion, as well as metastasis (see, e.g., Ma, P. C., Maulik, G., Christensen, J. & Salgia, R. (2003b). Cancer Metastasis Rev, 22, 309-25; Maulik, G., Shrikhande, A., Kijima, T., Ma, P. C., Morrison, P. T. & Salgia, R. (2002b). Cytokine Growth Factor Rev, 13, 41-59). c-MET can be activated through overexpression or mutations in various human cancers including small cell lung cancer (SCLC) (Ma, P. C., Kijima, T., Maulik, G., Fox, E. A., Sattler, M., Griffin, J. D., Johnson, B. E. & Salgia, R. (2003a). Cancer Res, 63, 6272-6281). Several c-MET inhibitors are known, including small molecule, ligand and antibody inhibitors (see references herein).

It would be desirable to have novel methods of treating abnormal cell growth, such as cancers, using such c-MET inhibitors in combination with other agents that enhance the efficacy of the c-MET inhibitors.

### SUMMARY OF THE INVENTION

In one embodiment, the invention provides a method of treating abnormal cell growth in a mammal, such as a human, by administering to the mammal a therapeutically effective amount of a c-MET inhibitor and an mTOR inhibitor.

mTOR is an important signaling intermediate molecule downstream of the PI3K/AKT pathway that inhibits apoptosis, and is important in nutritional status checkpoint (see, e.g., Grunwald, V., DeGraffenried, L., Russel, D., Friedrichs, W. E., Ray, R. B. & Hidalgo, M. (2002). Cancer Res, 62, 6141-5; Nave, B. T., Ouwens, M., Withers, D. J., Alessi, D. R. & Shepherd, P. R. (1999). Biochem J, 344 Pt 2, 427-31; Scott, P. H., Brunn, G. J., Kohn, A. D., Roth, R. A. & Lawrence, J. C., Jr. (1998). Proc Natl Acad Sci USA, 95, 7772-7; Stolovich, M., Tang, H., Hornstein, E., Levy, G., Cohen, R., Bae, S. S., Birnbaum, M. J. & Meyuhas, O. (2002). Mol Cell Biol, 22, 8101-13). mTOR is a large (Mᵣ ˜289,000) multidomain serine/threonine kinase, and is a member of the PI3K family of protein kinases based on homology within its catalytic domain.

Mammalian target of rapamycin (“mTOR”) regulates the activity of at least two proteins involved in the translation of specific cell cycle regulatory proteins. One of these proteins, p70s6 kinase, is phosphorylated by mTOR on serine 389 as well as threonine 412. This phosphorylation can be observed in growth factor treated cells by Western blotting of whole cell extracts of these cells with antibody specific for the phosphoserine 389 residue. As used herein, the term “mTOR inhibitor” means a compound or ligand which inhibits cell replication by blocking progression of the cell cycle from G1 to S by inhibiting the phosphorylation of serine 389 of p70s6 kinase by mTOR. One skilled in the art can readily determine if a compound, such as a rapamycin derivative, is an mTOR inhibitor. A specific method of making such determination is disclosed in U.S. patent application Publication No. 2003/0008923, the disclosure of which is incorporated herein by reference in its entirety.

A preferred mTOR inhibitor, rapamycin, is described in U.S. Pat. No. 3,929,992, the disclosure of which is incorporated herein by reference in its entirety. Rapamycin is also know by its USAN generic name, sirolimus.

As used herein, the term “rapamycin derivatives” includes compounds having the rapamycin core structure as defined in U.S. patent application Publication No. 2003/0008923, which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives.

Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No: 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5, 563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin derivatives” include those compounds and classes of compounds referred to as “rapalogs” in, for example, WO 98/02441 and references cited therein, and “epirapalogs” in, for example, WO 01/14387 and references cited therein, the disclosures of which are incorporated herein by reference in their entireties.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD.

Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, Protopic and Prograf.

Another preferred mTOR inhibitor is ABT-578 an antiproliferative agent (Abbott Laboratories). ABT-578 is believed to inhibit smooth muscle cell proliferation with a cytostatic effect resulting from the inhibition of mTOR.

Other preferred mTOR inhibitors include AP-23675, AP-23573, and AP-23841 (Ariad).

Preferred rapamycin derivatives include everolimus, CCI-779 [rapamycin 42-ester with 3-hydroxy-2-(hydroxymethyl)-2-methylpropionic acid; U.S. Pat. No. 5,362,718]; 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxy)ethyl rapamycin [U.S. Pat. No. 5,665,772].

In one embodiment, the c-MET inhibitor is a small molecule c-MET inhibitor. Examples of c-MET inhibitors include the 5-aralkylsulfonyl-3-(pyrrole-2ylmethylidene)-2-indolinone compounds disclosed in U.S. Pat. No. 6,599,902, and the compounds disclosed in WO 2001/60814, the disclosures of which are incorporated herein in their entireties. One skilled in the art can readily identify those compounds suitable as c-MET inhibitors by carrying out the assays as described, for example, in U.S. Pat. No. 6,599,902.

Preferred c-MET inhibitors include those having c-MET inhibitory activity as defined by any one or more of IC₅₀, Ki, or percent inhibition. One skilled in the art can readily determine if a compound has such activity by carrying out the appropriate assay. In one embodiment, particularly preferred compounds have a c-MET IC₅₀ of less than 5 μM, or less than 2 μM, or less than 1 μM, or less than 500 nM, or less than 400 nM, or less than 300 nM, or less than 200 nM, or less than 100 nM, or less than 50 nM. In another embodiment, particularly preferred compounds have a c-MET Ki of less than 5 μM or less than 2 μM, or less than 1 μM, or less than 500 nM, or less than 400 nM, or less than 300 nM, or less than 200 nM, or less than 100 nM, or less than 50 nM. In another embodiment, particularly preferred compounds have a c-MET inhibition at 1 μM of at least 10% or at least 20% or at least 30% or at least 40% or at least 50% or at least 60% or at least 70% or at least 80% or at least 90%. Methods of determining these c-MET activity values are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In one embodiment, the c-MET inhibitor is a compound of formula 1

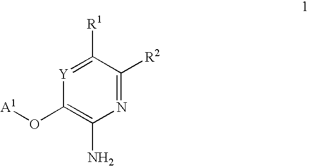

wherein:

Y is N or CR¹²;

R¹ is selected from C₆₋₁₂ aryl, 5-12 membered heteroaryl, C₃₋₁₂ cycloalkyl, 3-12 membered heteroalicyclic, —O(CR⁶R⁷)ₙR⁴ , —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₂₋₈ alkyl, C₂₋₈ alkenyl, and C₂₋₈ alkynyl; and each hydrogen in R¹ is optionally substituted by one or more R³ groups;

R² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R² is optionally substituted by one or more R⁸ groups;

R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each hydrogen in R³ is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;

each R⁴, R⁵, R⁶ and R⁷ is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; or any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;

each R⁸ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —CN, —O-C₁₋₁₂ alkyl, (CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;

A¹ is —(CR⁹R¹⁰)ₙ-A² except that:
(i) when Y is N and R¹ is substituted or unsubstituted aryl or substituted or unsubstituted heteroaryl, A¹ is —(CR⁹R¹⁰)ₙ-A² and n is not zero; and
(ii) when Y is N and R² is H and A¹ is m-chlorobenzyl, R¹ is not unsubstituted piperazine;

each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR₆R⁷)ₙC(O)OR⁴, —(CR₆R₇)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; and each hydrogen in R⁹ and R¹⁰ is optionally substituted by one or more R³ groups;

A² is C₆₋₁₂ aryl, 5-12 membered heteroaryl, C₃₋₁₂ cycloalkyl or 3-12 membered heteroalicyclic, and A² is optionally substituted by one or more R³ groups;

each R¹¹ is independently halogen, C₁₋₁₂ alkyl, C₁₋₁₂ alkoxy, C₃₋₁₂ cycloalkyl, C₆₋₁₂ aryl, 3-12 membered heteroalicyclic, 5-12 membered heteroaryl, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;

R¹² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR₆R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R¹² is optionally substituted by one or more R³ groups;

R¹ and R² or R¹ and R¹² may be combined together to form a C₆₋₁₂ aryl, 5-12 membered heteroaryl, C₃₋₁₂ cycloalkyl or 3-12 membered heteroalicyclic group;

m is 0, 1 or 2;

n is 0, 1, 2, 3 or 4; and

p is 1 or 2;

or a pharmaceutically acceptable salt, solvate or hydrate thereof.

In a particular aspect of this embodiment, Y is N. In a preferred aspect, R¹ is not piperazine. In another preferred aspect, R¹ is not heteroalicyclic.

In another particular aspect of this embodiment, Y is CR¹².

In particular aspects of this embodiment, and in combination with any other particular aspects of this embodiment, the compound has formula 1a

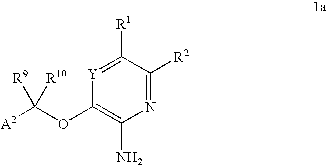

wherein A² is C₆₋₁₂ aryl or 5-12 membered heteroaryl optionally substituted by one or more R³ groups.

In particular aspects of this embodiment, and in combination with any other particular aspects of this embodiment, R¹ is selected from C₆₋₁₂ aryl and 5-12 membered heteroaryl, and each hydrogen in R¹ is optionally substituted by one or more R³ groups.

In particular aspects of this embodiment, and in combination with any other particular aspects of this embodiment not inconsistent with the following definition of RI, R¹ is selected from C₃₋₁₂ cycloalkyl, 3-12 membered heteroalicyclic, —O(CR⁶R⁷)ₙR⁴, —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups.

In particular aspects of this embodiment, and in combination with any other particular aspects of this embodiment, A² is substituted by at least one halogen atom.

In particular aspects of this embodiment, and in combination with any other particular aspects of this embodiment, R² is hydrogen, R⁹ and R¹⁰ are independently C₁₋₄ alkyl, and A² is phenyl substituted by at least one halogen atom.

In particular aspects of this embodiment, and in combination with any other particular aspects of this embodiment not inconsistent with the following definition of R¹, R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, piperazine, triazine, trithiane or phenyl group, and each hydrogen in R¹ is optionally substituted by one or more R³ groups.

In particular aspects of this embodiment, and in combination with any other particular aspects of this embodiment not inconsistent with the following definition of R¹, R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, triazine, trithiane or phenyl group, and each hydrogen in R¹ is optionally substituted by one or more R³ groups. In a more particular aspect, R¹ is not heteroalicyclic.

In particular aspects of this embodiment, and in combination with any other particular aspects of this embodiment not inconsistent with the following definition of R¹, R¹ is a fused ring heteroaryl group, and each hydrogen in R¹ is optionally substituted by one or more R³ groups.

In particular aspects of this embodiment, and in combination with any other particular aspects of this embodiment not inconsistent with the following definition of R¹, R¹ is a —SO₂NR⁴R⁵ group.

Specific compounds of this embodiment, and methods of synthesizing compounds of this embodiment, are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In another embodiment, the c-MET inhibitor is a compound of formula 2

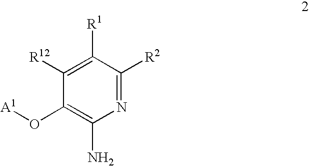

wherein:

R¹ is selected from C₆₋₁₂ aryl, 5-12 membered heteroaryl, C₃₋₁₂ cycloalkyl, 3-12 member heteroalicyclic, —O(CR⁶R⁷)ₙR⁴, —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups;

R² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R² is optionally substituted by one or more R⁸ groups;

R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each hydrogen in R³is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;

each R⁴, R⁵, R⁶ and R⁷ is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; or any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;

each R⁸ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —CN, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;

A¹is —(CR⁹R¹⁰)ₙ-A²;

each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR⁶R⁷)ₙC(O)OR⁴, —(CR⁶R⁷)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; and each hydrogen in R⁹ and R¹⁰ is optionally substituted by one or more R³ groups;

A² is C₆₋₁₂ aryl, 5-12 membered heteroaryl, C₃₋₁₂ cycloalkyl or 3-12 membered heteroalicyclic, and A² is optionally substituted by one or more R³ groups;

each R¹¹ is independently halogen, C₁₋₁₂ alkyl, C₁₋₁₂ alkoxy, C₆₋₁₂ cycloalkyl, C₆₋₁₂ aryl, 3-12 membered heteroalicyclic, 5-12 membered heteroaryl, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;

R¹² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R¹² is optionally substituted by one or more R³ groups;

R¹ and R² or R¹ and R¹² may be combined together to form a C₆₋₁₂ aryl, 5-12 membered heteroaryl, C₃₋₁₂ cycloalkyl or 3-12 membered heteroalicyclic group;

m is 0, 1 or 2;

n is 0,1,2,3 or 4; and

p is 1 or 2;

or a pharmaceutically acceptable salt, solvate or hydrate thereof.

In a particular aspect of this embodiment, the compound has formula 2a

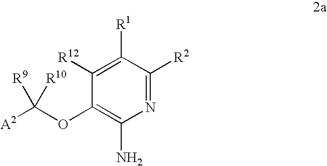

wherein A² is C₆₋₁₂ aryl or 5-12 membered heteroaryl optionally substituted by one or more R³ groups.

In particular aspects of this embodiment, and in combination with any other particular aspects of this embodiment, R¹ is selected from C₆₋₁₂ aryl and 5-12 membered heteroaryl, and each hydrogen in R¹ is optionally substituted by one or more R³ groups.

In particular aspects of this embodiment, and in combination with any other particular aspects of this embodiment not inconsistent with the following definition of R¹, R¹ is selected from C₃₋₁₂ cycloalkyl, 3-12 membered heteroalicyclic, —O(CR⁶R⁷)ₙR⁴, —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups.

In particular aspects of this embodiment, and in combination with any other particular aspects of this embodiment, A² is substituted by at least one halogen atom.

In particular aspects of this embodiment, and in combination with any other particular aspects of this embodiment, R² is hydrogen, R⁹ and R¹⁰ are independently C₁₋₄ alkyl, and A² is phenyl substituted by at least one halogen atom.

In particular aspects of this embodiment, and in combination with any other particular aspects of this embodiment not inconsistent with the following definition of R¹, R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, piperazine, triazine, trithiane or phenyl group, and each hydrogen in R¹ is optionally substituted by one or more R³ groups.

In particular aspects of this embodiment, and in combination with any other particular aspects of this embodiment not inconsistent with the following definition of R¹, R¹ is a fused ring heteroaryl group, and each hydrogen in R¹ is optionally substituted by one or more R³ groups.

In particular aspects of this embodiment, and in combination with any other particular aspects of this embodiment not inconsistent with the following definition of R¹, R¹ is a —SO₂NR⁴R⁵ group.

Specific compounds of this embodiment, and methods of synthesizing compounds of this embodiment, are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In another embodiment, the c-MET inhibitor is a compound of formula 3

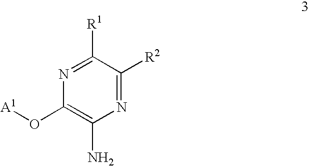

wherein:

R¹ is selected from C₆₋₁₂ aryl, 5-12 membered heteroaryl, C₃₋₁₂ cycloalkyl, 3-12 membered heteroalicyclic, —O(CR⁶R⁷)ₙR⁴, —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups;

R² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R²is optionally substituted by one or more R⁸ groups;

R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each hydrogen in R³is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;

each R⁴, R⁵, R⁶ and R⁷ is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; or any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;

each R⁸ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —CN, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;

A¹ is —(CR⁹R¹⁰)ₙ-A² except that:
(i) when R¹ is substituted or unsubstituted aryl or substituted or unsubstituted heteroaryl, A¹ is —(CR⁹R¹⁰)ₙ-A² and n is not zero; and
(ii) when R² is H and A¹ is m-chlorobenzyl, R¹ is not unsubstituted piperazine;

each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR⁶R⁷)ₙC(O)OR⁴, —(CR⁶R⁷)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; and each hydrogen in R⁹ and R¹⁰ is optionally substituted by one or more R³ groups;

A² is C₆₋₁₂ aryl, 5-12 membered heteroaryl, C₃₋₁₂ cycloalkyl or 3-12 membered heteroalicyclic, and A² is optionally substituted by one or more R³ groups;

each R¹¹ is independently halogen, C₁₋₁₂ alkyl, C₁₋₁₂ alkoxy, C₃₋₁₂ cycloalkyl, C₆₋₁₂ aryl, 3-12 membered heteroalicyclic, 5-12 membered heteroaryl, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;

R¹ and R² may be combined together to form a C₆₋₁₂ aryl, 5-12 membered heteroaryl, C₃₋₁₂ cycloalkyl or 3-12 membered heteroalicyclic group;

m is 0, 1 or 2;

n is 0, 1, 2, 3 or4; and

p is 1 or 2;

or a pharmaceutically acceptable salt, solvate or hydrate thereof.

In a particular aspect of this embodiment, the compound has formula 3a

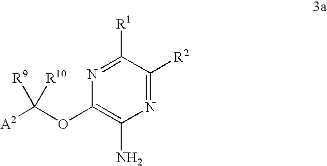

wherein A² is C₆₋₁₂ aryl or 5-12 membered heteroaryl optionally substituted by one or more R³ groups.

In particular aspects of this embodiment, and in combination with any other particular aspects of this embodiment, R¹ is selected from C₆₋₁₂ aryl and 5-12 membered heteroaryl, and each hydrogen in R¹ is optionally substituted by one or more R³ groups.

In particular aspects of this embodiment, and in combination with any other particular aspects of this embodiment not inconsistent with the following definition of R¹, R¹ is selected from C₃₋₁₂ cycloalkyl, 3-12 membered heteroalicyclic, —O(CR⁶R⁷)ₙR⁴, —C(O)R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups.

In particular aspects of this embodiment, and in combination with any other particular aspects of this embodiment, A² is substituted by at least one halogen atom.

In particular aspects of this embodiment, and in combination with any other particular aspects of this embodiment, R² is hydrogen, R⁹ and R¹⁰ are independently C₁₋₄ alkyl, and A² is phenyl substituted by at least one halogen atom.

In particular aspects of this embodiment, and in combination with any other particular aspects of this embodiment not inconsistent with the following definition of R¹, R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, piperazine, triazine, trithiane or phenyl group, and each hydrogen in R¹ is optionally substituted by one or more R³ groups.

In particular aspects of this embodiment, and in combination with any other particular aspects of this embodiment not inconsistent with the following definition of R¹, R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, triazine, trithiane or phenyl group, and each hydrogen in R¹ is optionally substituted by one or more R³ groups. In still more particular aspects, R¹ is not heteroalicyclic.

In particular aspects of this embodiment, and in combination with any other particular aspects of this embodiment not inconsistent with the following definition of R¹, R¹ is a fused ring heteroaryl group, and each hydrogen in R¹ is optionally substituted by one or more R³ groups.

In particular aspects of this embodiment, and in combination with any other particular aspects of this embodiment not inconsistent with the following definition of R¹, R¹ is a —SO₂NR⁴R⁵ group.

Specific compounds of this embodiment, and methods of synthesizing compounds of this embodiment, are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In another embodiment, the c-MET inhibitor is a compound of formula 4

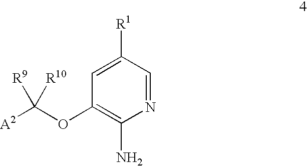

wherein:

R¹ is selected from C₆₋₁₂ aryl, 5-12 membered heteroaryl, C₃₋₁₂ cycloalkyl, 3-12 membered heteroalicyclic, —O(CR⁶R⁷)ₙR⁴, —C(O)R⁴, —C(O)OR⁴, —CN, —NO₂, —S(O)ₘR⁴, —SO₂N R⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen is R¹ is optionally substituted by one or more R³ groups;

R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, 13 OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each hydrogen in R³is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;

each R⁴, R⁵, R⁶ and R⁷is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;

each R⁸ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —CN, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;

each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR⁶R⁷)ₙC(O)OR⁴, —(CR⁶R⁷)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; and each hydrogen in R⁹ and R¹⁰ is optionally substituted by one or more R³ groups;

A² is C₆₋₁₂ aryl, 5-12 membered heteroaryl, C₃₋₁₂ cycloalkyl or 3-12 membered heteroalicyclic, and A² is optionally substituted by one or more R³ groups;

each R¹¹ is independently halogen, C₁₋₁₂ alkyl, C₁₋₁₂ alkoxy, C₃₋₁₂ cycloalkyl, C₆₋₁₂ aryl, 3-12 membered heteroalicyclic, 5-12 membered heteroaryl, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;

m is 0, 1 or 2;

n is 0,1, 2, 3 or 4; and

p is 1 or 2;

or a pharmaceutically acceptable salt, solvate or hydrate thereof.

In a particular aspect of this embodiment, A² is C₆₋₁₂ aryl or 5-12 membered heteroaryl optionally substituted by one or more R³ groups.

In other particular aspects of this embodiment, preferred substituents and groups of substituents include those defined in particular aspects of the previous embodiments.

Specific compounds of this embodiment, and methods of synthesizing compounds of this embodiment, are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In another embodiment, the c-MET inhibitor is a compound of formula 5

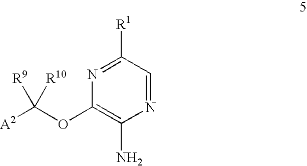

wherein:

R¹ is selected from C₆₋₁₂ aryl, 5-12 membered heteroaryl, C₃₋₁₂ cycloalkyl, 3-12 membered heteroalicyclic, —O(CR⁶R⁷)ₙR⁴, —C(O) R⁴, —C(O)OR⁴, —CN, —NO₂, —S(O)ₘR⁴, —SO₂NR⁴R⁵, —C(O)NR⁴R⁵, —NR⁴C(O)R⁵, —C(═NR⁶)NR⁴R⁵, C₁₋₈ alkyl, C₂₋₈ alkenyl, and C₂₋₈ alkynyl; and each hydrogen in R¹ is optionally substituted by one or more R³ groups;

R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each hydrogen in R³ is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;

each R⁴, R⁵, R⁶ and R⁷ is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; or any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;

each R⁸ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —CN, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;

each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR⁶R⁷)ₙC(O)R⁴, —(CR⁶R⁷)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; and each hydrogen in R⁹ and R¹⁰ is optionally substituted by one or more R³ groups;

A² is C₆₋₁₂ aryl, 5-12 membered heteroaryl, C₃₋₁₂ cycloalkyl or 3-12 membered heteroalicyclic, and A² is optionally substituted by one or more R³ groups; except that when R², R⁹ and R¹⁰ are all H and A² is m-chlorophenyl, R¹ is not unsubstituted piperazine;

each R¹¹ is independently halogen, C₁₋₁₂ alkyl, C₁₋₁₂ alkoxy, C₃₋₁₂ cycloalkyl, C₆₋₁₂ aryl, 3-12 membered heteroalicyclic, 5-12 membered heteroaryl, —O—C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;

m is 0, 1 or 2;

n is 0, 1, 2, 3 or 4; and

p is 1 or 2;

or a pharmaceutically acceptable salt, solvate or hydrate thereof.

In a particular aspect of this embodiment, A² is C₆₋₁₂ aryl or 5-12 membered heteroaryl optionally substituted by one or more R³ groups.

In other particular aspects of this embodiment, preferred substituents and groups of substituents include those defined in particular aspects of the previous embodiments.

Specific compounds of this embodiment, and methods of synthesizing compounds of this embodiment, are described in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

In another embodiment, the c-MET inhibitor is selected from the group consisting of the compounds of Tables 1-6 of WO 04/076412, and their pharmaceutically acceptable salts.

In another embodiment, the c-MET inhibitor is selected from the group consisting of 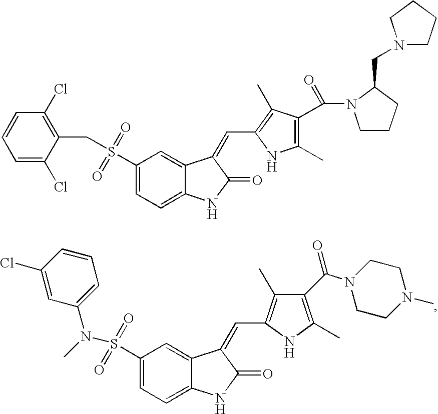
and their pharmaceutically acceptable salts. These two compounds are described, including their synthesis, in U.S. Pat. Nos. 6,599,902 and 6,573,293, respectively. The disclosures of these two patents are incorporated herein by reference in their entireties.

In another embodiment, the c-MET inhibitor is a c-MET antibody. Examples of c-MET antibodies include those disclosed in U.S. Pat. No. 6,468,529, and U.S. Provisional Patent Application No. 60/492432, filed Aug. 4, 2003, the disclosures of which are incorporated herein by reference in their entireties. A preferred c-MET antibody is 5D5 FAb, described in U.S. Pat. No. 6,468,529.

In another embodiment, the c-MET inhibitor is a c-MET ligand antagonist. Examples of c-MET ligand antagonists include the HGF fragment NK4 of Kringle Pharma. NK4 is described in K. Date et al., “HGF/NK4 is a specific antagonist for pleiotrophic actions of hepatocyte growth factor,” FEBS Lett.,420: 1-6 (1997); K. Date et al., “Inhibition of tumor growth and invasion by a four-kringle antagonist (HGF/NK4) for hepatocyte growth factor,” Oncogene 17: 3045-3054 (1998); K. Kuba et al., “HGF/NK4, a four-kringle antagonist of hepatocyte growth factor, is an angiogenesis inhibitor that suppress tumor growth and metastasis in mice,” Cancer Res. 60: 6737-6743 (2000); K. Kuba et al., “Kringle 1-4 of hepatocyte growth factor inhibits proliferation and migration of human microvascular endothelial cells,” Biochem. Biophys. Res. Commun. 279: 846-852 (2000); D. Tomioka et al., “Inhibition of growth, invasion, and metastasis of human pancreatic carcinoma cells by NK4 in an orthotopic mouse model,” Cancer Res. 61: 7518-7524 (2001); Japan Patent Application No. JP 300728/1995 to Nakamura (Osaka Univ.), filed Oct. 24, 1995, entitled “Anti-Cancer Agent” and corresponding international application no. PCT/JP96/03105, filed Oct. 23, 1996; and Japan Patent Application No. JP 134681/98 to Nakamura (Osaka Univ.), filed Apr. 28, 1998, entitled “Neovascularization Inhibitors” and corresponding international application no. PCT/JP99/01834. The disclosures of these references are incorporated herein in their entireties.

It should be appreciated that combinations of any of the mTOR inhibitors described herein with any of the c-MET inhibitors described herein are within the scope of the invention.

In a specific embodiment of any of the inventive methods described herein, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In further specific embodiments of any of the inventive methods described herein, the method further comprises administering to the mammal an amount of one or more substances selected from anti-tumor agents, anti-angiogenesis agents, signal transduction inhibitors, and antiproliferative agents, which amounts are together effective in treating said abnormal cell growth. Such substances include those disclosed in PCT publication nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 240 -N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethyinitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2, 4-diamino-5-methyl-6-quinazolinyl)methyl]amino]benzoyl]-L-aspartic acid, N-[4-[[(2, 4-diamino-5-ethyl-6-quinazolinyl)methyl]amino]benzoyl]-L-aspartic acid, N -[2-chloro-4-[[(2,4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadexm (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17,1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), United States Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of MMP inhibitors include AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published January 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, New Jersey, USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined or co-administered with the composition. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26,1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody bevacizumab (Genentech, Inc. of South San Francisco, Calif.); and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with the composition. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in United States Provisional Application No. 60/117,341, filed Jan. 27, 1999, and in United States Provisional Application No. 60/117,346, filed Jan. 27,1999, both of-which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following U.S. patent application Ser. No. 09/221946 (filed Dec. 28, 1998); Ser. No. 09/454058 (filed Dec. 2, 1999); Ser. No. 09/501163 (filed Feb. 9, 2000); Ser. No. 09/539930 (filed Mar. 31, 2000); 09/202796 (filed May 22, 1997); Ser. No. 09/384339 (filed Aug. 26, 1999); and Ser. No. 09/383755 (filed Aug. 26, 1999); and the compounds disclosed and claimed in the following U.S. provisional patent applications: 60/168207 (filed Nov. 30, 1999); 60/170119 (filed Dec. 10, 1999); 60/177718 (filed Jan. 21, 2000); 60/168217 (filed Nov. 30,1999), and 60/200834 (filed May 1, 2000). Each of the foregoing patent applications and provisional patent applications is herein incorporated by reference in their entirety.

The composition may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

Specific examples of combination therapy can be found in PCT Publication No. WO 03/015608 and U.S. Provisional Patent Application No. 60/426,386, filed Nov. 15, 2002, the disclosures of which are incorporated herein by reference in their entireties.

In another embodiment, the invention provides a pharmaceutical composition comprising a c-MET inhibitor and an mTOR inhibitor, wherein the c-MET inhibitor is any of the c-MET inhibitors described herein and the mTOR inhibitor is any of the mTOR inhibitors described herein.

In another embodiment, the invention provides administering a pharmaceutical composition comprising a c-MET inhibitor and an mTOR inhibitor, wherein the c-MET inhibitor is any of the c-MET inhibitors described herein and the mTOR inhibitor is any of the mTOR inhibitors described herein, in any of the methods described herein.

### DEFINITIONS

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

As used herein, “administering” refers to the delivery of a compound or salt of the present invention or of a pharmaceutical composition containing a compound or salt of this invention to an organism for the purpose of prevention or treatment of abnormal cell growth.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

### BRIEF DESCRIPTION OF THE DRAWINGS

FIG. 1 shows PHA665752 inhibition of cell growth of TPR-MET transformed BaF3 cells.

FIG. 2 shows PHA665752-induced apoptosis and cell cycle arrest in TPR-MET transformed BaF3 cells.

FIG. 3 is a schematic diagram of the functional domain structure and the tyrosine phospho-sites of the wild type c-MET and the oncogenic fusion TPR-MET.

FIG. 4 shows that PHA665752 inhibits MET-mediated tyrosine phosphorylation and TPR-MET autophosphorylation, and regulates cell growth through an mTOR-dependent pathway.

FIG. 5 shows that PHA665752 cooperates with rapamycin in regulating growth through an mTOR-dependent pathway.

### DETAILED DESCRIPTION OF THE INVENTION

Administration of the c-MET inhibitor and the mTOR inhibitor can be effected by any method that enables delivery of the compounds to the site of action. These methods include oral routes, intraduodenal routes, parenteral injection (including intravenous, subcutaneous, intramuscular, intravascular or infusion), topical, and rectal administration. The c-MET inhibitor and the mTOR inhibitor are administered to the patient as part of course of treatment that includes treatment with both types of inhibitors. The specific dosing regimen for the c-MET inhibitor and the mTOR inhibitor can be the same or different, as can the specific dosage form. One skilled in the art can readily determine appropriate dosage forms and dosing regimens. If desired, the c-MET inhibitor and the mTOR inhibitor can be provided as a single dosage form including both inhibitors. Alternatively, the dosage forms can be distinct and need not be the same type of dosage form. Thus, by way of an illustrative example only, one of the inhibitors may be administered twice daily in a suspension formulation, and the other of the inhibitors may be administered once daily by tablet.

The inhibitor may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository. The inhibitor may be in unit dosage forms suitable for single administration of precise dosages. Preferably, dosage forms include a conventional pharmaceutical carrier or excipient and the c-MET inhibitor and/or the mTOR inhibitor as an active ingredient. In addition, dosage forms may include other medicinal or pharmaceutical agents, carriers, adjuvants, etc.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The examples and preparations provided below further illustrate and exemplify the methods of the present invention. It is to be understood that the scope of the present invention is not limited in any way by the scope of the following examples.

### EXAMPLES

The following abbreviations are used in the Examples herein: DMSO, dimethysulfoxide; FCS, fetal-calf serum; GIST, gastrointestinal stromal tumor; HGF, hepatocyte growth factor/scatter factor; IC₅₀, concentration for 50% inhibitory effect; IL-3, IL-3; JM, juxtamembrane; mTOR, mammalian target of rapamycin; PDGFR, platelet-derived growth factor receptor; pY or pTyr, phosphotyrosine; PBS, phosphate buffered saline; PI3K, phosphatidylinositol-3′-kinase; RTK, receptor tyrosine kinase; TBS, Tris buffered saline; TBST, TBS plus Tween 20.

Materials and Methods:

An exemplary c-MET inhibitor, denoted PHA665752 or (3Z)-5-[(2,6-dichlorobenzyl)sulfonyl]-3-[(3,5-dimethyl-4-{[(2R)-2-(pyrrolidin-1-ylmethyl)pyrrolidin-1-yl]carbonyl}-1H-pyrrol-2-yl)methylene]-1,3-dihydro-2H-indol-2-one, was used. This compound has the structural formula 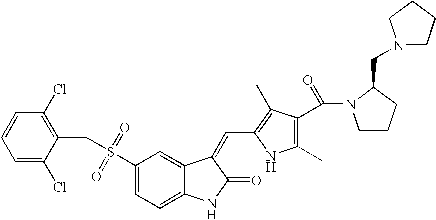
and is described, including its synthesis, in U.S. Pat. No. 6,599,902, the disclosure of which is incorporated herein by reference in its entirety.

An exemplary mTOR inhibitor, rapamycin (Calbiochem, La Jolla, Calif.) was used in the following examples. The c-MET inhibitor and mTOR inhibitor were dissolved in DMSO and used at the indicated concentrations.

Cells. The murine pre-B cell line BaF3 was grown in RPMI 1640 containing 10% fetal calf serum and 10% WEHI-conditioned medium as a source of murine IL-3. BaF3 cell lines transfected with a BCR/ABL, TEUABL, TEUJAK2, or TEUPDGFβR cDNA were grown in the absence of growth factors. A TPR/MET expressing BaF3 cell line was generated by transfection of an expression vector containing the TPR/MET cDNA as previously described (Sattler, M., Pride, Y. B., Ma, P., Gramlich, J. L., Chu, S. C., Quinnan, L. A., Shirazian, S., Liang, C., Podar, K., Christensen, J. G. & Salgia, R. (2003). Cancer Res, 63, 5462-9.). The number of viable cells after treatment with DMSO or PHA665752 was determined using an MTT assay (In Vitro Toxicology Assay Kit, Sigma, St. Louis, Mo.) or trypan blue exclusion.

Transwell migration assay. The lower chamber of a transwell plate (8 μm pore size polycarbonate membrane, Corning Costar Corp., Cambridge, Mass.) was filled with 600 μL starvation media (0.5%, w/v, BSA in RPMI 1640). Cells were counted using a Coulter particle counter (Coulter Counter Z2, Beckman Coulter, Fullerton, Calif.) and resuspended at 2×10⁶ cells/mL in starvation media. 100 μL of this cell suspension was transferred to the upper chamber. The medium contained either PHA665752 (0.2 μM) or DMSO in the control samples. After 4 hours, cells in the lower compartment were resuspended and counted using a Coulter particle counter. The spontaneous transwell migration of cells was expressed as a “migration index” (number of migrating cells treated with PHA665752 divided by the number of migrating cells left untreated). The standard error of the mean was calculated from the migration indices of independently performed experiments. The statistical significance of the data was analyzed using the Student's t-test.

Immunoblotting. Proteins were extracted from whole cells by lysing them in a Tris buffer (50 mM, pH 8.0) containing NaCl (150 mM), NP40 (1%, v/v), deoxycholic acid (0.5%, w/v), sodium dodecylsulfate (0.1%, w/v), NaF (1 mM), Na₃VO₄ (1 mM) and glycerol (10%, v/v) (Sigma, St. Louis, Mo.) supplemented with a protease inhibitor cocktail (complete, Roche, Indianapolis, Ind.). Polyclonal antibodies against p70-S6K (Biosource International, Camarillo, Calif.), total c-MET (C-12, Santa Cruz, Santa Cruz, Calif.), phosphatidylinositol-3′-kinase (Upstate Biotechnology, Lake Placid, N.Y.) and phosphorylated AKT[Ser473] or p70-S6K[Thr421/Ser424] (Cell Signaling, Beverly, Mass.), phospho-MET[Tyr1230/1234/1235] (Biosource International, Camarillo, Calif.) as well as phosphotyrosine (4G10, Upstate Biotechnology, Lake Placid, N.Y.) were used for immunoblotting.

Apoptosis assays. The activity of caspase-3-was measured in cell lysates (CaspACE Assay System, Promega) and Annexin V positive staining was determined by FACS analysis (Annexin-V-Fluos Staining Kit, Roche Diagnostics) according to the manufacturer's directions in cells that were either treated with PHA665752 or the solvent DMSO.

Cell cycle analysis. Fixed cells were stained with propidiumiodide and cell cycle parameters analyzed by FACS analysis.

### Example 1

This example shows that the small molecule c-MET inhibitor PHA665752 specifically regulates cell growth in TPR-MET transformed BaF3 cells.

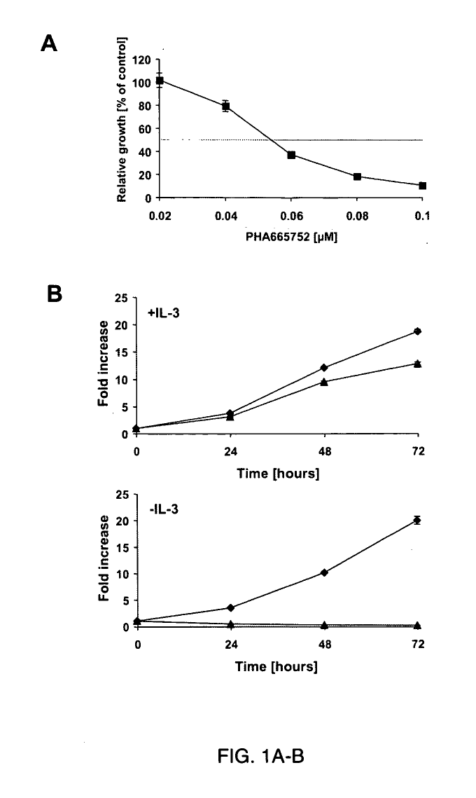

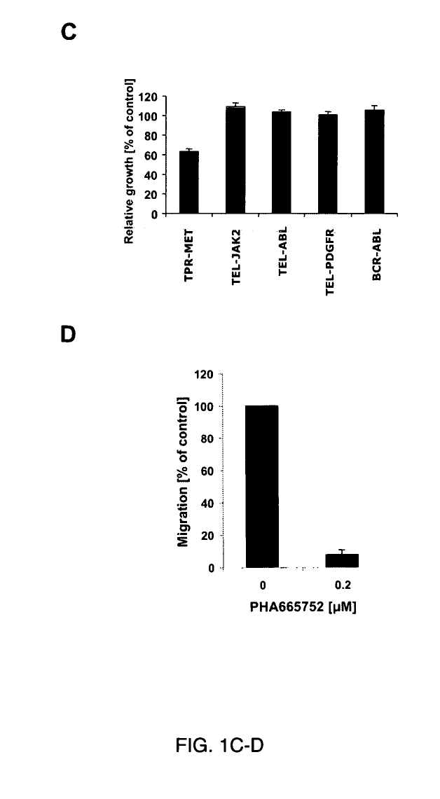

PHA665752 was identified as a prototype ATP-competitive small molecule inhibitor of the catalytic kinase activity of the MET RTK. We initially sought to determine if PHA665752 could inhibit cell growth in TPR-MET transformed BaF3 cells (FIG. 1A). Treatment of BaF3.TPR-MET cells with PHA665752 was found to inhibit cell growth in a dose dependent manner with an IC₅₀<0.06 μM. To further determine if the growth inhibitory effect of PHA665752 on BaF3 TPR-MET cells accumulates over time, the cell growth was determined over a 72 hour culture. In the presence of IL-3, PHA665752 had only little effect on cell growth of TPR-MET transformed cells or BCR-ABL transformed cells in a control experiment (FIG. 1B, top panel). In contrast, PHA665752 completely blocked cell growth in the absence of IL-3 in BaF3. TPR-MET and even reduced the number of viable cells (FIG. 1B, bottom panel). This suggests that IL-3 partially rescues the BaF3.TPR-MET cells from PHA665752-dependent growth inhibition. We did not observe a significant growth inhibitory effect of PHA665752 at 0.2 μM, in IL-3 stimulated parental BaF3 cells in a 72 hour culture (data not shown). TPR-MET is therefore implicated in the deregulation of pathways normally utilized by the activated IL-3 receptor, similar to the relation between the Abl inhibitor STI-571 and the BCR-ABL oncoprotein. PHA665752 (0.2 μM, 18 hours) also did not inhibit cell growth of BaF3 cells transformed by other oncogenic tyrosine kinases, including BCR-ABL, TEL-JAK2, TEL-ABL and TEL-PDGFFBR (FIG. 1C).

Untransformed BaF3 cells do not migrate through a transwell membrane. However, when transformed by TPR-MET, the cells display spontaneous transwell migration with enhanced cell motility. In addition to cell growth, PHA665752 was also found to inhibit this aspect of transformation (FIG. 1D). Migration of BaF3.TPR-MET cells was inhibited with 0.2 μM PHA665752 (92.5±3% inhibition of the cell migration) compared to DMSO treated cells. This demonstrates that the TPR-MET kinase activity regulates cell growth, motility and migration of the transformed BaF3 cells.

Referring to FIG. 1, BaF3 cells lines transformed by tyrosine kinase oncogenes were used to determine cell growth (A-C) or transwell migration (D) in response to the small molecule c-MET kinase inhibitor PHA665752. A: The relative growth of BaF3 cells transformed by TPR-MET in response to different concentrations of PHA665752 was determined after 18 h (n=3). B: TPR-MET transformed BaF3 cells were either left untreated (♦) or treated (▴) with PHA665752 (1 μM) for the indicated time in the presence or absence of IL-3 (n=3). C: BaF3 cells transformed by tyrosine kinase oncogenes were treated for 18 h with PHA665752 (1 μM) (n=3). D: Cells were treated for 18 hr with the indicated dose of PHA665752 and the spontaneous transwell migration relative to DMSO-treated cells determined (n=4).

### Example 2

This example shows that inhibition of MET kinase activity by PHA665752 induces apoptosis and cell cycle arrest in TPR-MET transformed BaF3 cells.

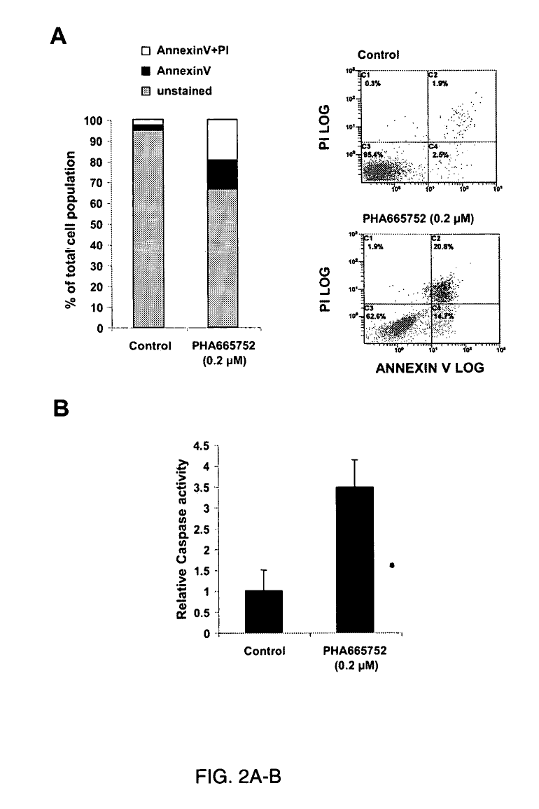

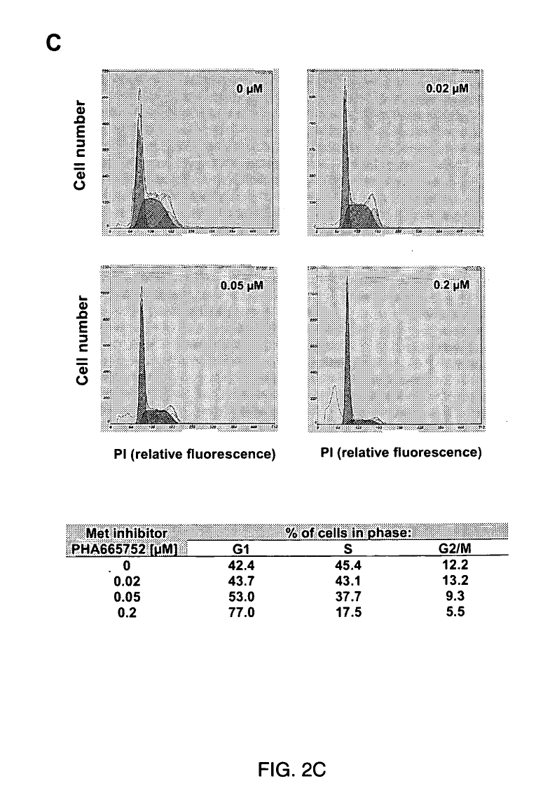

Apoptosis is a complex cellular function that is regulated in part through the c-MET tyrosine kinase activity in TPR-MET transformed cells and inhibition of c-MET kinase is therefore expected to induce an increase in apoptosis. We measured the change in Annexin V positive staining of cells, an indication for increased exposure of phosphatidylserine to the outer cell membrane during apoptosis. Using TPR-MET transformed BaF3 cells, we found that treatment with PHA665752 (0.2 □M, 18 h) led to an increase in Annexin V positive cells compared to DMSO treated cells (FIG. 2A, top left). In the control cells, 5% of the total population showed signs of apoptosis, however, the number of apoptotic cells increased to 33.1% after PHA665752 treatment. On average 13.9±1.0% of the cells were in early apoptosis (Annexin V positive) and 19.2±1.8% of the cells were in late apoptosis (Annexin V plus propidiumiodide positive). We thereafter measured the activation status of caspase-3, a downstream effector of the pro-apoptotic caspase-9. Similar to the previous data, we observed a consistent increase in caspase-3 activity (3.5±0.7 fold increase; n=3; p<0.03) compared to DMSO treated cells (FIG. 2B).

We also determined if inhibition of the TPR-MET tyrosine kinase would induce cell cycle arrest. Cells were treated with DMSO or different amounts of the c-MET kinase inhibitor and the different phases of cell cycle distribution were then determined (FIG. 2C). The percentage of cells in G1-phase increased from 42.4% to 77.0% in PHA665752 (0.2 μM) treated cells, whereas the percentage of cells in S-phase (reduced from 45.4% to 17.5%) and G2/M-phase (reduced from 12.2% to 5.5%) decreased. This suggests that inhibition of TPR-MET kinase activity leads to G1 cell cycle arrest in the transformed cells. In addition, there was an increase of cells in sub-G1-phase, which was consistent with apoptotic cells. These data demonstrate that PHA665752 induces cell cycle arrest as well as apoptosis, and both events in combination are likely to contribute to the reduced cell growth of the PHA665752-treated TPR-MET transformed cells.

Referring to FIG. 2, TPR-MET transformed BaF3 cells were treated for 18 h with either DMSO or the indicated amount of PHA665752 (n=3). A: Annexin V and propidiumiodide staining was determined by flow cytometry. B: Activity of caspase-3 was determined in cell lysate (n=3). C: The percentage of cells in different cell cycle phases was determined by flow cytometry after propidiumiodide staining (n =3).

### Example 3

This example shows that PHA665752 inhibits tyrosine phosphorylation of cellular proteins in TPR-MET transformed BaF3 cells.

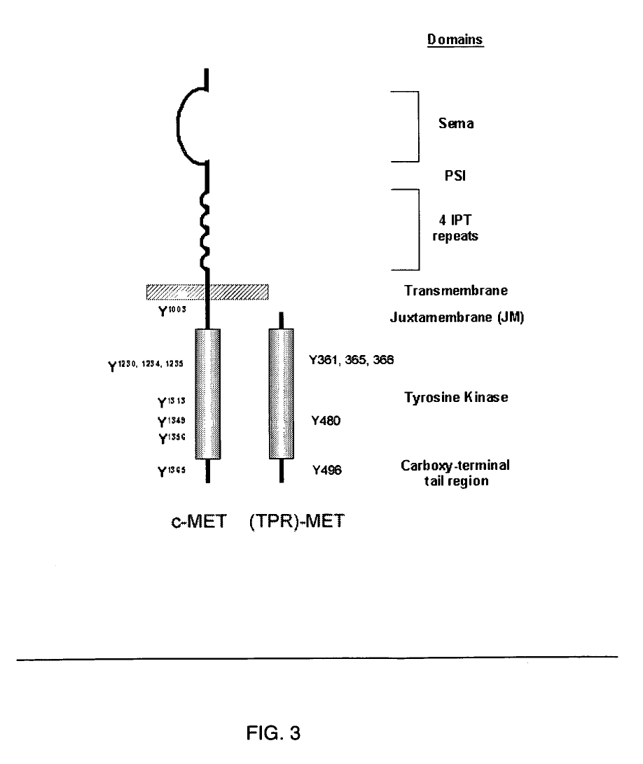

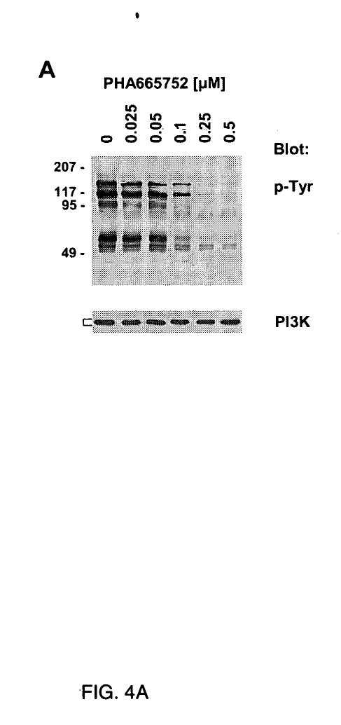

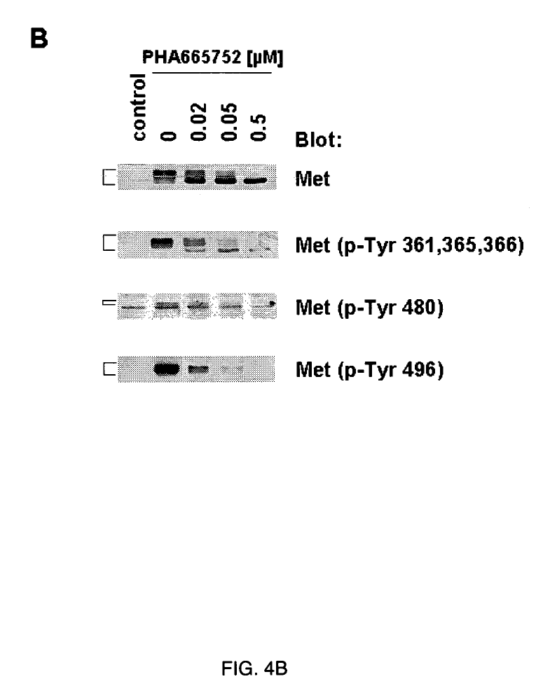

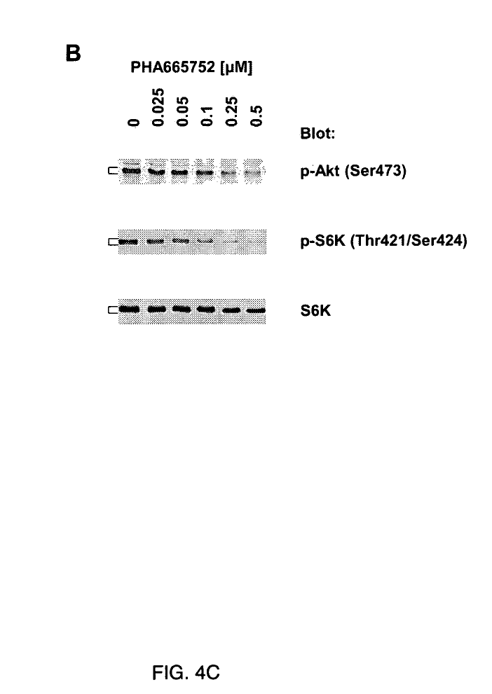

FIG. 3 is a schematic diagram of the functional domain structure and the tyrosine phospho-sites of the wild type c-MET and the oncogenic fusion TPR-MET. Wild type c-MET is composed of the large extracellular sema domain, which harbors the HGF- and heparin-binding sites, the PSI and four IPT repeats; followed by the transmembrane and the cytoplasmic juxtamembrane domain and the catalytic tyrosine kinase domain. TPR-MET (TPR not shown) contains only the cytoplasmic portion of c-MET with the juxtamembrane domain missing. The corresponding tyrosine phosphorylation sites of c-MET and TPR-MET are also shown here In order to determine the biochemical consequences of MET kinase inhibition by PHA665752 in BaF3.TPR-MET cells, changes in tyrosine phosphorylation of cellular proteins were evaluated. The tyrosine phosphorylation sites in TPR-MET with the corresponding sites in the tyrosine kinase domain of c-MET are shown schematically in FIG. 3. The juxtamembrane domain of c-MET is deleted as a result of the chromosomal translocation resulting in the TPR-MET fusion oncoprotein. Treatment of BaF3.TPR-MET cells with PHA665752 reduced tyrosine phosphorylation of cellular proteins in a dose dependent manner (FIG. 4A), but did not alter tyrosine phosphorylation of cellular proteins in BCR-ABL transformed BaF3 cells (data not shown). These data are consistent with the dose-dependent reduction of cell growth shown above and suggest that PHA665752 specifically inhibits TPR-MET induced tyrosine phosphorylation relative to BCR-ABL. Also, using phosphospecific antibodies against tyrosine phosphorylation sites in c-MET, we found that PHA665752 inhibits autophosphorylation in the catalytic tyrosine kinase domain at Tyr361/365/366 (autophosphorylation site), Tyr480 (Grb2 binding site) and Tyr496 (important in cell morphogenesis) (FIG. 4B).

In addition, we sought to determine if inhibition of TPR-MET would reduce the phosphorylation and alter the activation status of pathways that are involved in cell growth and proliferation. We found that the dose-dependent reduction in tyrosine phosphorylation of cellular proteins after PHA665752 treatment correlated with reduced serine phosphorylation of AKT[Ser473] as well as the reduced phosphorylation of the mTOR substrate p70-S6K[Thr421/Ser424] (FIG. 4C). This would suggest that inhibition of MET kinase activity leads to reduced activation of the phosphatidylinositol-3′-kinase AKT/mTOR pathway in these transformed cells.

Referring to FIG. 4, phosphorylation of cellular proteins was determined by immunoblotting in whole cell lysate as indicated using anti-phosphotyrosine antibody (4G10) (A), total c-MET antibody, anti-pY1230/1234/1235-MET antibody (recognizing the corresponding pY361/365/366 sites in TPR-MET), anti-pY1349-MET (recognizing the pY480 site in TPR-MET) and anti-pY1365-MET (recognizing the pY496 site in TPR-MET) phospho-antobodies (B), and phospho-AKT and phospho-S6K antibodies (C). TPR-MET transformed BaF3 cell were treated with the indicated amount of PHA665752. Blots were probed for equal loading with antibodies against p85 PI3K or p70-S6K (A-C).

### Example 4

This example shows that PHA665752 cooperates with rapamycin to inhibit cell growth in TPR-MET transformed BaF3 cells through a mTOR-dependent pathway.

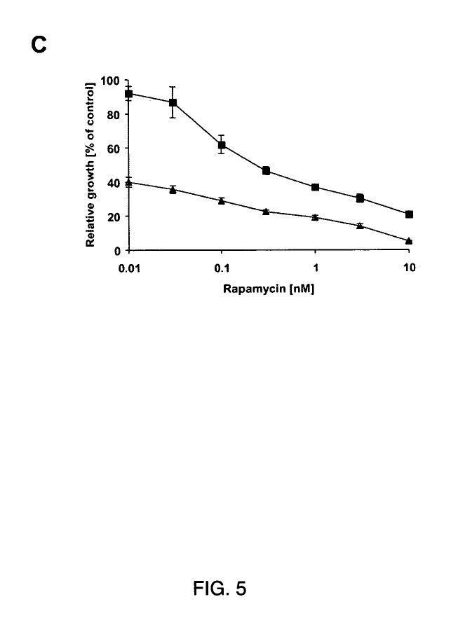

We determined the significance of mTOR regulation by c-MET in the cells with the specific mTOR inhibitor rapamycin. In the absence of PHA665752, rapamycin reduced cell growth of the BaF3.TPR-MET cells in a dose-dependent manner. In the presence of PHA665752 (0.05 μM), rapamycin cooperated with the c-MET inhibitor in inhibiting cell growth of the TPR-MET transformed cells (FIG. 5). This suggests that PHA665752 acts in part by inhibiting the mTOR pathway and that rapamycin or related drugs may well be suited for combination therapy.

Referring to FIG. 5, the relative growth of BaF3 cells transformed by TPR-MET in response to different concentrations of rapamycin (0.01 nM to 10 nM) was determined in the presence (▴) or absence (▪) of PHA665752 (0.5 μM) after a 3 day culture (n=3).

### Tables

Specific examples of small molecule c-MET inhibitors include the compounds in U.S. Provisional Patent Application No. 60/449,588, filed Feb. 26, 2003, and U.S. Provisional Application No. 60/540,229, filed Jan. 29, 2004, published as WO 04/076412, the disclosures of which are incorporated herein by reference in their entireties.

All references cited herein, including priority documents, are incorporated by reference herein in their entireties.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

All references cited herein, including any priority documents, are hereby incorporated by reference in their entireties.

We claim:

1. A method of treating abnormal cell growth in a mammal, the method comprising administering to the mammal a therapeutically effective amount of a c-MET inhibitor and an mTOR inhibitor.

2. The method of claim 1, wherein the mTOR inhibitor is selected from the group consisting of rapamycin and derivatives thereof.

3. The method of claim 1, wherein the mTOR inhibitor is selected from the group consisting of rapamycin, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, and 42-O-(2-hydroxy)ethyl rapamycin.

4. The method of claim 1,wherein the c-MET inhibitor is a c-MET antibody.

5. The method of claim 1, wherein the c-MET inhibitor is a c-MET ligand antagonist.

6. The method of claim 1, wherein the c-MET inhibitor is a compound of formula 1 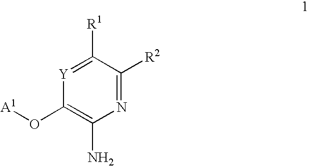
wherein:
Y is N or CR¹²;
R¹ is selected from C₆₋₁₂ aryl, 5-12 membered heteroaryl, C₃₋₁₂ cycloalkyl, 3-12 membered heteroalicyclic, —O(CR⁶R⁷)ₙR⁴, —C(O) R⁴, —C(O)OR⁴, —CN, —NO₂, —S(O)ₘR⁴, —SO₂N R⁴R⁵, —C(O)NR⁴R⁵, —NR⁴C(O)R⁵, —C(═NR⁶)NR⁴R⁵, C₁₀₈ alkyl, C₂₋₈ alkenyl, and C₂₋₈ alkynyl; and each hydrogen is R¹ is optionally substituted by one or more R³ groups;
R² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)R⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R² is optionally substituted by one or more R⁸ groups;
R³ is halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴, —(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, each group in R³ is optionally substituted by one or more R⁸ groups, and R³ groups on adjacent atoms may combine to form a C₆₋₁₂ aryl, 5-12 membered heteroaryl, C₃₋₁₂ cycloalkyl or 3-12 membered heteroalicyclic group;
each R⁴, R⁵, R⁶ and R⁷ is independently hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl; or any two of R⁴, R⁵, R⁶ and R⁷ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O, and S; or any two of R⁴, R⁵, R⁶ and R⁷ bound to the same carbon atom may be combined to form a C₃₋₁₂ cycloalkyl, C₆₋₁₂ aryl, 3-12 membered heteroalicyclic or 5-12 membered heteroaryl group; and each hydrogen in R⁴, R⁵, R⁶ and R⁷ is optionally substituted by one or more R⁸ groups;
each R⁸ is independently halogen, C₁₋₁₂ alkyl, C₂₋₁₂ alkenyl, C₂₋₁₂ alkynyl, C₃₋₁₂ cycloalkyl, C₁₋₁₂ aryl, 3-12 membered heteroalicyclic, 5-12 membered heteroaryl, —CN, —O—-C₁₋₁₂ alkyl, —O—(CH₂)ₙC₃₋₁₂ cycloalkyl, —O—(CH₂)ₙC₆₋₁₂ aryl, —O—(CH₂)ₙ(3-12 membered heteroalicyclic) or —O—(CH₂)ₙ(5-12 membered heteroaryl); and each hydrogen in R⁸ is optionally substituted by one or more R¹¹ groups;
A¹ is —(CR⁹R¹⁰)ₙ-A² except that:
(i) when Y is N and R¹ is substituted or unsubstituted aryl or substituted or unsubstituted heteroaryl, A¹ is —(CR⁹R¹⁰)ₙ-A² and n is not zero; and
(ii) when Y is N and R² is H and A¹ is m-chlorobenzyl, R¹ is not unsubstituted piperazine;
each R⁹ and R¹⁰ is independently hydrogen, halogen, C₁₋₁₂ alkyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —NR⁴C(O)R⁵, —(CR⁶R⁷)ₙC(O)OR⁴, —(CR₆R₇)ₙNCR⁴R⁵, —NR⁴C(O)NR⁵R⁶, —NR⁴S(O)ₚR⁵ or —C(O)NR⁴R⁵; R⁹ and R¹⁰ may combine to form a C₃₋₁₂ cycloalkyl, 3-12 membered heteroalicyclic, C₆₋₁₂ aryl or 5-12 membered heteroaryl ring; each hydrogen in R⁹ and R¹⁰ is optionally substituted by one or more R³ groups;
A² is C₆₋₁₂ aryl, 5-12 membered heteroaryl, C₃₋₁₂ cycloalkyl or 3-12 membered heteroalicyclic, and A² is optionally substituted by one or more R³ groups;
each R¹¹ is independently halogen, C₁₋₁₂ alkyl, C₁₋₁₂ alkoxy, C₃₋₁₂ cycloalkyl, C₆₋₁₂ aryl, 3-12 membered heteroalicyclic, 5-12 membered heteroaryl, —O—C₁₋₂ alkyl, —O—(CH₂)ₙC₃-₁₂ cycloalkyl, —O—(CH₂)ₙC₆₋₁₂ aryl, —O—(CH₂)ₙ(3-12 membered heteroalicyclic), —O—(CH₂)ₙ(5-12 membered heteroaryl) or —CN, and each hydrogen in R¹¹ is optionally substituted by one or more groups selected from halogen, —OH, —CN, —C₁₋₁₂ alkyl which may be partially or fully halogenated, —O—C₁₋₁₂ alkyl which may be partially or fully halogenated, —CO, —SO and —SO₂;
R¹² is hydrogen, halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heteroalicyclic, 5-12 membered heteroaryl, —S(O)ₘR⁴, —SO₂NR⁴R⁵, —S(O)₂OR⁴, —NO₂, —NR⁴R⁵, —(CR⁶R⁷)ₙOR⁴, —CN, —C(O)R⁴, —OC(O)R⁴, —O(CR⁶R⁷)ₙR⁴, —NR⁴C(O)R⁵, —(CR⁶R⁷)ₙC(O)OR⁴—(CR⁶R⁷)ₙNCR⁴R⁵, —C(═NR⁶)NR⁴R⁵, —NR⁴C(O)NR⁵R⁶, —NR⁴S(O)ₚR⁵ or —C(O)NR⁴R⁵, and each hydrogen in R¹² is optionally substituted by one or more R³ groups;
R¹ and R² or R¹ and R¹² may be combined together to form a C₆₋₁₂ aryl, 5-12 membered heteroaryl, C₃₋₁₂ cycloalkyl or 3-12 membered heteroalicyclic group;
m is 0, 1 or 2;
n is 0, 1, 2, 3 or 4; and
p is 1 or 2;
or a pharmaceutically acceptable salt, solvate or hydrate thereof.

7. The method of claim 1, wherein the c-MET inhibitor is selected from the group consisting of 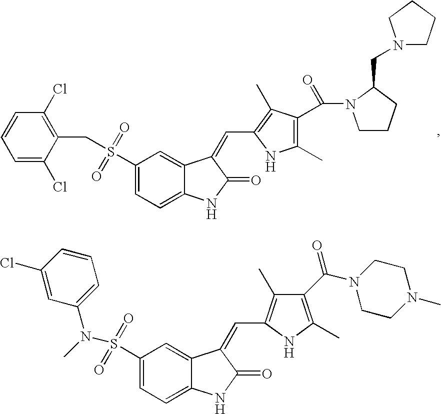
and pharmaceutically acceptable salts thereof.

8. The method of claim 1, wherein the abnormal cell growth is cancer.

9. The method of claim 8, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

10. The method of claim 1, wherein the method further comprises co-administering an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, anti-androgens and mixtures thereof.

11. The method of claim 1, wherein the c-MET inhibitor and the mTOR inhibitor are administered as separate dosage forms.

12. The method of claim 1, wherein the c-MET inhibitor and the mTOR inhibitor are administered to the mammal as a single dosage form.

13. A pharmaceutical composition comprising a therapeutically effective amount of a c-MET inhibitor and an mTOR inhibitor.
